(19) United States
(12) Patent Application Publication
(10) Pub. No.: US 2012/0308616 A1
(43) Pub. Date: Dec. 6, 2012

(54) SUBMICRO EMULSION OF PACLITAXEL USING STEROID COMPLEX AS INTERMEDIATE CARRIER

(75) Inventors: Yuling Liu, Beijing (CN); Xuejun Xia, Beijing (CN); Ruifang Guo, Beijing (CN); Pengxiao Zhang, Beijing (CN); Rui Han, Beijing (CN); Zhaodi Fu, Beijing (CN); Cuiping Zhou, Beijing (CN); Renyun Wang, Beijing (CN); Dujia Jin, Beijing (CN)

(73) Assignee: INSTITUTE OF MATARIA MEDICA, CHINESE ACADEMY OF MEDICAL SCIENCES, Beijing (CN)

(21) Appl. No.: 13/505,168

(22) PCT Filed: Oct. 28, 2010
(86) PCT No.: PCT/CN10/78209

§ 371 Date: Aug. 21, 2012

(30) Foreign Application Priority Data
Oct. 29, 2009 (CN) 200910236959.4

(51) Int. Cl. A61K 9/107; A61P 35/00; A61K 31/575; B82Y 5/00

(52) U.S. Cl. 424/400; 514/171; 977/788; 977/915

(57) ABSTRACT

A submicron emulsion of paclitaxel, the preparation method and the use thereof are disclosed. Said paclitaxel submicron emulsion comprises paclitaxel/steroid complex, oil for injection, water for injection, emulsifier, assistant emulsifier and isotonic agent, wherein the mole ratio of paclitaxel to steroid in the complex is 1:0.2˜4; preferably 1:0.25˜2. Said submicron emulsion is useful for the treatment for malignant tumor. The average particle diameter of the submicron emulsion is less than 400 nm and the pH Value is 3.5-6.

### FIELD OF THE INVENTION

The present invention relates to a paclitaxel submicron emulsion using steroid complex as an intermediate carrier and its preparation procedure, and also the application of this paclitaxel submicron emulsion, which belongs to the field of pharmaceutical preparation technology.

### BACKGROUND OF THE INVENTION

Paclitaxel (paditaxel, Taxol) possesses an important anti-tumor activity, thus has been widely used in the treatment of ovarian and breast cancer, non-small cell lung cancer (NSCLC), head and neck carcinoma in clinical practice. Since it is barely soluble in water (0.006 μg/ml), the common paclitaxel injection formulation Taxol® presently used in clinical practice is prepared through dissolving 30 mg paclitaxel into 5 ml mixture of Cremopher EL (ethoxylate castor oil)/alcohol (50:50, V/V). Because of the large amount of Cremopher EL in the formulation, it tends to stimulate the release of histamine in vivo, resulting in severe allergic reactions. With regard to this, the following desensitization is required before clinical use: 10 mg oral dexamethasone is given 12 hours before treatment, and a second dose of 10 mg oral dexamethasone is given 6 hours before treatment, and 30 to 60 minutes before treatment, diphenhydramine, 20 mg, i.m., cimetidine, 300 mg, i.v. or ranitidine, 50 mg, i.v. is given. Despite these precautions, 5% to 30% of the patients may still experience allergies at different degrees in clinical practice. Furthermore, there may be physical stability issue once the concentrated paclitaxel solution solubilized by Cremopher EL/alcohol is diluted, for example, the drug may precipitate due to a low temperature or a long instilling time, thus patients' safety may be at risk.

Given the undesired effects of the paclitaxel injection formulation, pharmaceutical scientists both in China and worldwide have performed studies on a new formulation system since paclitaxel was launched more than 20 years ago, and technological strategies include formulations made by using cyclodextrin inclusion complex, liposome, polymeric micelle, nanoparticle and so on.

Although cyclodextrin inclusion complex could increase paclitaxel's solubility, cyclodextrin used at large quantities could cause severe renal toxicity; also the drug may precipitate once dilution is performed through water, therefore, this type of formulation has not been implemented in clinical practice so far.

Liposome has disadvantages including low entrapment efficiency, being prone to leakage if stored for a long time and precipitation after dilution through water, thus it is difficult to develop this type of formulation commercially and no product of this category is even though there has been on-going investment abroad for 20 years. The paclitaxel liposome (lipusu) freeze-dried power for injection includes 30 mg of the drug in each, its specification and dose for clinical use is identical to common injections available, and the efficacy is not significantly different, however, a preparation procedure is introduced and a pretreatment for desensitization is also needed, therefore, it is not technologically superior.

There are plenty of studies concerning paclitaxel polymeric micelle on-going both domestically and abroad, but its industrialization is limited by low drug loading, unstable quality after storage and requirement for lyophilization during storage. In the past years, research relating to polymeric micelle is developing very fast due to the sprouting of new polymer materials, however the medicinal safety of introducing large amounts of polymeric materials with completely new structures needs further evaluation.

The protein-bound paclitaxel nanoparticle injection (manufactured by Bioscience, Inc.) approved by FDA in 2005 is so far the most important new patented paclitaxel formulation all over the world, and it is designed to use human plasma albumin as a carrier and to prepare the paclitaxel formulation as protein-bound nanoparticles, which is made into freeze-dried power for injection after aseptic filtration, freezing and drying. Compared to common injections, the albumin-bound paclitaxel nanoparticle formulation for injection is superior in the following aspects: 1) this formulation is Cremopher EL free, thus allergic reaction is completely avoided, which makes it the only new paclitaxel formulation requiring no desensitization treatment worldwide; 2) due to its low toxicity and high tolerance, the clinical used dosage for patients is increased from 135-175 mg/m² to 260 mg/m², thus resulting in a significantly better clinical efficacy than common paclitaxel injections. However, due to the large amount of the carrier, albumin, which is extremely expensive (up to 6200 Yuan for each injection), as well as its highly complicated and strict preparation procedures, the clinical use of albumin-bound paclitaxel nanoparticle is very limited.

The oil-in-water submicron emulsion is an emulsion of particles with an average diameter less than 600 nm obtained through homogeneous emulsification under high pressure using natural phospholipid as emulsifier after dissolving the medicine into the oil phase and the basis is the drug's lipotropy. Because inside the medicine there exists an inner oil phase, which avoids direct contact between water and air, thus overcoming the difficulty in preparing liquid formulations of drugs having low solubility and stability. Compared to liposome technology, submicron emulsion is more convenient to industrialize; and compared to albumin bound nanoparticle, an oil-in-water submicron emulsion has a lower manufacturing cost, could be sterilized at terminal, can be injected directly in clinical practice, does not tend to precipitate, and is safe and convenient to administrate. Therefore, there is a promising future to develop a new paclitaxel formulation using a submicron emulsion as carrier. Although both domestic and oversee scholars have done lots of experiments on paclitaxel submicron emulsions, the drug loading in the submicron emulsion manufactured through conventional procedures is under 0.02 mg/ml due to paclitaxel's low solubility in water as well as an extremely low solubility in oil; moreover, the medicine may transfer from the oil phase into the water phase during disinfection and storage, resulting in demulsification, stratification and concentration. Restricted by its low solubility in water, no paclitaxel submicron emulsion with high drug loading that is tolerant to sterilization under heat and pressure and stable through long-term storage has been developed in the world.

In order to improve paclitaxel's solubility in oil phase and ease the restriction from its physicochemical properties on the development of submicron emulsion, we performed preliminary studies on “Paclitaxel Liposome Complex” and “Paclitaxel Submicron Emulsion Using Liposome as Intermediate Carrier”, and applied for two patents: application No. CN200810168213.X, “Paclitaxel Liposome Complex” and application No. CN200810168212.5, “Paclitaxel Submicron Emulsion Using Liposome as Intermediate Carrier”.

For the paclitaxel liposome complex disclosed in patent application CN200810168213.X, natural egg yolk lecithin, granulesten and cholesterol were carefully chosen as the liposome material, and the proportion of paclitaxel and liposome material is 1:1˜19 by weight, i.e. the amount of liposome is up to 1˜19 times of that of paclitaxel (more specifically, for phospholipid, the mole ratio between paclitaxel and liposome is 1:1˜20; for cholesterol, it is 1:2.2˜20; for bile acids, it is 1:2.1˜40). The submicron emulsion formulation disclosed in patent application CN200810168212.5 adopts the paclitaxel liposome complex in patent application CN200810168213.X as the intermediate carrier.

The paclitaxel liposome complex is designed to improve the solubility of paclitaxel in oil and provide qualified intermediate carrier for subsequent manufacture of submicron emulsion. However, through further investigation, the following problems are identified in the technological protocol mentioned in patent applications CN200810168213.X and CN200810168212.5.

1. Although the drug solubility in oil could be significantly improved by using phospholipid to prepare the complex, the maximum is limited to 2 mg/ml, and the solubility in oil is not further increased by adding more phospholipid. Limited by low solubility in oil, the maximum drug loading is restricted to 0.5 mg/ml if submicron emulsion is prepared by using liposome complex as the intermediate carrier, and the entrapment efficiency is under 80%, there is obvious stratification after storage up to 6 months, thus it could not meet the requirements of medical treatment; with a drug loading up to 1.0 mg/ml, it could not form even emulsion.

2. Cholesterol could significantly improve the drug solubility in oil than phospholipid when used as the liposome material for the complex. However, cholesterol is a steroid, which could result in various disadvantages since its amount is 1-19 times of that of paclitaxel: (1) overdose: a healthy adult intakes about 300 mg˜500 mg cholesterol each day (equivalent to the cholesterol in 1˜2 eggs), and one medicinal dose of paclitaxel is 300 mg, as for the cholesterol complex and its formulation involved in patent application CN200810168213.X, the cholesterol intake is about 300 mg˜5700 mg, with the highest dosage equivalent up to 19 egg yolks, which is significantly excessive and could lead to safety risk; (2) instability of the submicron emulsion prepared through long-term storage: if cholesterol complex is used as the intermediate carrier during submicron emulsion preparation, based on the medicinal formulation and specific paclitaxel concentration, higher the liposome material is used in the complex, more complex will be encapsulated inside the inner oil phase in the submicron emulsion. Restricted by the volume of the oil drop inside the oil phase and interface between oil and water, when the amount of complexes encapsulated exceeds that content that the oil phase and the interface between oil and water, part of the drug may be driven to the outer water phase, resulting in a low entrapment efficiency and instability of the submicron emulsion prepared. Through investigation of the submicron emulsion described in patent application CN200810168212.5, the entrapment efficiency is 65% to 85%, and the quality is essentially stable if stored for 6 months at 4° C.; However, there is obvious stratification when it is stored up to 12 months, the declared content drops and the paclitaxel impurity is significantly increased; (3) high manufacture cost: factors including large amounts of liposome used, solvent largely used during manufacture and long duration taken to evaporize the solvent, result in high manufacture cost, which disobeys the principle of pharmacological economy.

For purposes of providing a submicron emulsion with low lipid content, high entrapment efficiency and good stability, after extensive research, this disclosure provides a submicron emulsion prepared using a steroid complex with low lipid content as an intermediate carrier. In the steroid complex, the molar ratio of paclitaxel/cholesterol is 1:0.2˜4, preferably 1:0.25˜2, best 1:0.33˜1; correspondingly, the ratio between paclitaxel and cholesterol by weight is 1:0.09˜1:1.94, preferably is 1:0.11˜1:0.97, and best 1:0.15˜1:0.49. Compared to patent application CN200810168212.5, the complex in this application manages a complete paclitaxel incorporation while significantly reducing the use of steroid, thus increasing the solubility of drug in the oil as it possibly can. The solubility tends to remain stable while the steroid is further increased, indicating no further increasing effect. Since the steroid complex could improve the solubility of the drug in the oil, the complex is dissolved into the oil phase and a paclitaxel submicron emulsion with high entrapment efficiency, stable quality through long-term storage and low cholesterol contents is obtained using emulsifier and assistant emulsifier; thus the present invention is completed.

### Contents of the Invention

One purpose of the present invention is to provide a paclitaxel submicron emulsion, which consists of a paclitaxel/steroid complex, oil for injection, an emulsifier, an assistant emulsifier and an isotonic agent; the steroid in the paclitaxel/steroid complex as described is a natural steroid or one of its derivatives; the natural steroid as described is selected from the group consisting of cholesterol, 7-hydrocholesterol (also named 7-dehydrocholesterol), lanosterol, sitosterol, brassicasterol, mycosterol, ostreasterol, stigmasterol, sitosterolum and ergosterol, preferably cholesterol, 7-hydrocholesterol and ergosterol, more preferably cholesterol; the natural steroid derivatives as described are choosed from cholic acid, deoxycholic acid and anthropodesoxycholic acid.

The mean diameter of the submicron emulsion droplet in the present invention is under 400 nm, preferably under 300 nm; the ratio of oil phase is 5%˜35% (ml/ml) total amount of the submicron emulsion as described, preferably 10%˜30% (ml/ml); measured by paclitaxel, the drug load is 0.25 mg/ml˜5 mg/ml, preferably 0.5 mg/ml˜2 mg/ml.

In the present invention, the paclitaxel/steroid complex as described is prepared through the following procedure 1 or 2. Procedure 1 includes:

a. Mix paclitaxel and steroid at a specific ratio, add an appropriate volume of an organic solvent, choose any antioxidative stabilizer and add in;

b. Stir at a proper temperature, remove the organic solvent through rotating evaporation or sponge drying, and after vacuum drying, the complex is obtained.

Procedure 2 includes:

a. Prepare paclitaxel and steroid at a specific ratio, add appropriate volumes of different organic solvents, respectively, and then mix them, choose any antioxidative stabilizer and add in;

b. Stir at a proper temperature, remove the organic solvent through rotating evaporation or sponge drying, and after vacuum drying, the complex is obtained.

In the paclitaxel/steroid complex and its preparation procedures as described above, the mole ratio between paclitaxel and steroid is 1:0.2˜4, preferably 1:0.25˜2, the best 1:0.33˜1; correspondingly, the ratio between paclitaxel and steroid by weight is 1:0.09˜1:1.94, preferably 1:0.11˜1:0.97, and more preferably is 1:0.15˜1:0.49.

In the preparation procedures of paclitaxel/steroid as described above, the organic solvent as described is selected from the group consisting of methylene dichloride, alcohol, methanol, phenylcarbinol, acetone, ethyl acetate, tetrahydrofuran, tertiary butyl alcohol; for preferably, one or more is selected from the group consisting of alcohol, acetone, ethyl acetate and tetrahydrofuran. By “appropriate volume of organic solvent”, the “appropriate volume” means the volume that the technician in this filed could determine based on common specification for dissolving the mixture of paclitaxel and steroid, to be specific, the concentration of paclitaxel and steroid complex in the solution calculated by paclitaxel should be controlled to 0.5˜16 mg/ml or higher, preferably is 1.0˜8.0 mg/ml; “proper temperature” refers to 10° C.-70° C., preferably 35˜55° C., for example 5° C., 35° C., 45° C., 55° C. or 70° C. Both the duration of agitation reaction and vacuum drying could be determined by technicians in this area based on common specification, for example, the time for agitation reaction could be 0.5-3.0 hours, such as 0.5, 1.0, 1.5 or 2.0 hours, and the time for vacuum drying could be 8-48 hours, such as 8, 12, 16 or 24 hours. The antioxidative stabilizer as described is selected from the group consisting of sodium bisulfate, sodium metabisulfite, vitamin C, EDTA and its salt, vitamin E and its derivatives, and the amount of antioxidative stabilizer is the common amount used during the preparation of liposome complex in this area, and it usually does not exceed 1% (by weight) of the total amount of the complex.

In the present invention, the oil for injection as described is one or mixture from long chain or medium chain oil. The long chain oil as described is selected from the group consisting of long chain fatty acid, long chain fatty ester or long chain fatty alcohol, to be specific, one selected from the group consisting of soybean oil, castor oil, linoleic acid, maize oil, olive oil, oil of groundnuts, cotton seed oil, oleic acid, glyceryl monostearate, glycerol monooleate, cetanol; the medium chain oil is one selected from the group consisting of medium chain fatty acid and medium chain fatty ester. A preferred long chain oil is long chain fatty ester, specifically soybean oil for injection; the optimal selection for medium chain oil is long chain fatty acid glyceride.

In the present invention, the emulsifier as described is a nonionic surfactant or a natural surfactant. Nonionic surfactant is selected from the group consisting of fatty acid glyceride, polyoxyethylene fatty alcohol ethers, polyoxyethylene sorbitan monoacid esters, sorbitol and sorbitan fatty acid ester, polyoxyethylene fatty acid ethers, vitamin E derivatives and polyoxyolefin copolymer; the natural surfactant is selected from the group consisting of egg yolk lecithin, fabaceous lecithin, ornitrol and cholic acids, sodium alginate and chitosan. A preferred emulsifier is a natural surfactant, and a more preferred emulsifier is natural egg yolk lecithin and soybean lecithin. The content of the emulsifier in the submicron emulsion of the present invention is 0.5%-5% (g/ml) of the total submicron emulsion complex, preferably 1.0%-4.0% (g/ml), and more preferably 1.0%-2.0% (g/ml).

In the present invention, the assistant emulsifier as described is selected from the group consisting of polyethyleneglycol (PEG) and poloxamer. The content of the assistant emulsifier is 0%-5% (g/ml) of the total amount of the submicron emulsion in the present invention, preferably 0.5%-3% (g/ml), and more preferably 1.0%-2.0% (g/ml).

In the present invention, the osmo-regulator (isotonic agent) of the paclitaxel submicron emulsion is selected from the group consisting of glycerin, xylitol, sorbierite and mannitol, preferably glycerin and glucose, more preferably glycerin. The content of glycerin is 1.0-3.0% (g/ml) of the total amount of paclitaxel submicron emulsion in the present invention, preferably 1.5-2.5% (g/ml).

In the present invention, the paclitaxel submicron emulsion as described could add stabilizer, which could be one selected from the group consisting of among oleic acid, eunatrol and PEGs, preferably oleic acid and PEG, more preferably oleic acid. The content of oleic acid is 0.05-0.5% (g/ml) of the total amount of paclitaxel submicron emulsion in the present invention, preferably 0.1-0.2% (g/ml).

The paclitaxel submicron emulsion in the present invention could also include an antioxidant, which could be vitamin E or vitamin E ester derivatives, preferably vitamin E.

In the present invention, “one or mixture” or “at least one” means it could be one material being chosen, or a mixture of two or more of them.

Another purpose of the present invention is to provide a preparation procedure for the paclitaxel submicron emulsion as described in the present invention, including the following steps:
Prepare water for injection, add emulsifier, assistant emulsifier and isotonic agent, get a homogeneous water phase by dispersing in a tissue disintegrator or shear, heat up to 40-80° C., and maintain the temperature;
Obtain a paclitaxel/steroid complex, choose any stabilizer and dissolve both of them into the oil for injection preheated up to 40-80° C., obtain a homogeneous oil phase by dispersing in a tissue disintegrator or shear;
Under constant stirring, add the oil phase into the water phase slowly, mix for 5-10 min at 10000-20000 r/min to obtain a preliminary emulsion, and transfer it into a high pressure homogenizer quickly, particles with diameter under 400 nm are obtained through homogeneous emulsification, preferably under 300 nm, collect all the emulsion, adjust its pH to 3.5-6.0 through hydrochloric acid, preferably 4.0-5.0, add appropriate amount of water (to full volume), thus the product is obtained.

The present invention also provides another preparation procedure for the paclitaxel submicron emulsion, including the following steps:
Prepare water for injection, add assistant emulsifier and isotonic agent, get a water phase by stirring, heat up to 40-80° C., and insulate;
Get a paclitaxel/steroid complex, emulsifier and/or stabilizer, dissolve them into the oil for injection, get an even oil phase by dispersing in a tissue disintegrator or shear;
Under constant stirring, add the water phase into the oil phase slowly, mix for 5-10 min at 10000-20000 r/min to obtain a preliminary emulsion, and transfer it into a high pressure homogenizer quickly, particles with diameter under 400 nm are obtained through homogeneous emulsification, preferably 100-300 nm, collect all the emulsion, adjust its pH to 3.5-6.0 through hydrochloric acid, preferably 4.0-5.0, add appropriate amount of water (to full volume), thus the product is obtained.

In the preparation procedure of the present invention, emulsification in the high pressure homogenizer could be replaced by other emulsification measures as long as an even emulsification could be performed and the particle diameter as described is realized; the temperature, 40-80° C., could be 40° C., 50° C., 60° C., 70° C. or 80° C.

In the preparation procedure of the present invention, the concentration for the hydrochloric acid used to adjusting the pH could be a commonly used in this area, such as 0.1 mol/L or 0.01 mol/L; the amount of water and oil for injection could be determined by technicians in this field according to the ratio of the oil phase provided by the present invention, and technicians in this field could add water according to common specifications to get the amount of water corresponding to the drug loading provided by the present invention.

The present invention also provides a formulation, which includes the paclitaxel submicron emulsion and could be made into any preparation which could be clinically used, including infusion solution or dry emulsion. Wherein the infusion solution is prepared by the following procedure: after filling the paclitaxel submicron emulsion described in the present invention, an aseptic process is performed through circulating steam sterilization or steam sterilization, and the target preparation is obtained; wherein the dry emulsion is prepared by the following procedure: add appropriate amount of support agent into the paclitaxel submicron emulsion described in the present invention, after an aseptic process, dry emulsion is obtained through freeze-drying process. The preferred support agent is mannitol, such as 5% (w/v) mannitol.

The present invention also provides the application of the paclitaxel submicron emulsion of the present invention in the preparation of anticancer drugs, and the cancer as described can be a solid tumor, including oophoroma, breast cancer, cervical carcinoma, non-small cell lung cancer, head or neck cancer, esophagus cancer, renal carcinoma, liver cancer and gastric cancer.

Unless otherwise specified, all the scientific and technological terminology and name used in the present invention means the same as the understanding of common technicians in the field that the present invention belongs to; furthermore, if not specified, the material and its content or proportion, equipment, instrument, preparing condition are all those that the technicians in this field are familiar with or could understand based on the description in the present invention.

The paclitaxel submicron emusion provided by the present invention has the following special advantages:

1) Low manufacture cost and steroid intake: the paclitaxel submicron emulsion of the present invention uses a steroid complex with low lipid content as the intermediate carrier, since the amount of steroid in the complex is just 0.09˜1.94 of the paclitaxel by weight (preferably 0.11˜0.97), thus this patent application greatly reduces liposome material used, manufacture cost and risks caused by high dose of steroids compared to “Paclitaxel Submicron Emulsion Using Liposome as Intermediate Carrier” disclosed in patent application CN200810168212.5,

2) Good entrapment efficiency and stability: single dose of clinically used paclitaxel is 135˜175 mg/m², i.e. 240˜300 mg a dose per person. In addition, based on the submicron emulsion volume that human body could accept at one time, the volume of a single dose of paclitaxel submicron emulsion should be within the range of 100-500 ml. Calculated according to the foregoing, the concentration of paclitaxel in the submicron emulsion should fall in the range of 0.48 mg/ml-3.0 mg/ml. In “Paclitaxel Submicron Emulsion Using Liposome as Intermediate Carrier” disclosed in CN200810168212.5, the submicron emulsion formulation prepared using phospholipid complex as the intermediate carrier, the drug load is 0.5 mg/ml at most, and it is unstable after storage; comparing to this, the submicron emulsion formulation prepared using cholesterol complex as the intermediate carrier has a higher drug load, however, the amount of cholesterol is up to 1˜19 times of paclitaxel, consequently the total amount of complex encapsulated in the paclitaxel submicron emulsion is up to 1.2 mg/ml-60 mg/ml. As for the submicron emulsion formulation using steroid complex as the intermediate carrier in this disclosure, since the amount of steroid used is only 0.09˜1.94 of that of paclitaxel (preferably 0.11˜0.97), the total amount of complex encapsulated in the emulsion is only 1.14 mg/ml-3.60 mg/ml (preferably 1.39 mg/ml-1.80 mg/ml), thus, the formulation in the present invention has superior stability and is rather safe to human body.

As for the oil-in-water submicron emulsion formulation, the lipid soluble drug exists in the oil phase and/or the interface between oil and water, the volume of the oil droplet inside the oil phase and the volume of the interface are limited, the entrapment of the emulsion drops as the total amount of complexes used increase, therefore more drug goes into the outer water phase and the stability of the submicron emulsion after long-term storage will decrease. Compared to the technology disclosed in CN200810168212.5, in this disclosure, the total amount of the complexes encapsulated in the oil phase significantly decreases due to the use of a steroid complex with low lipid content as the intermediate carrier, in this way, the entrapment efficiency of the submicron emulsion is increased, the free drug in the water phase is reduced and the stability of the formulation is improved.

Comparison studies show that the entrapment efficiency of the submicron emulsion disclosed in 200810168212.5 is between 65%-85%, stratification appears after storage under refrigeration (4° C., the same below) up to 12 months, the drug content decreases, the impurity increases from preliminary 1% to 3.5%˜7.6%; as for the submicron emulsion in this disclosure, the entrapment efficiency is above 90%, no stratification is found after storage under refrigeration up to 12 months, the appearance is evenly adularescent, there is no obvious change with regard to the diameter or content of the particles, total impurity is less than 1.7%, within the range of preferable drug load, the entrapment efficiency is above 95%, the total impurity is under 1%, and the quality is stable.

3) Good safety and high tolerated dosage: compared to the common injection formulation presently available, the submicron emulsion provided in the present invention is alcohol and Cremopher EL free, which eases the vascular stimulation of paclitaxel formulation, prevents potential allergic reactions and toxicity and side effects caused by Cremopher EL, thus, the safety is improved and the tolerated dosage is increased, providing a solid basis for increasing dosage and improving efficacy. When nude mice are injected with one dosage every 3 days for 3 times, the maximum tolerated dose (without animal death) for the common injection formulation is 20 mg/kg, while for the submicron emulsion in this disclosure is 45 mg/kg, and the tolerated dose increases to 2.25 times and is identical to that of the albumin-bound paclitaxel nanoparticle formulation reported in literature (increasing to 2.23 times of that of common injections). By comparing the anticancer activity under tolerated doses, the result shows that the submicron emulsion of this disclosure has the highest tumor inhibition rate among breast cancer, oophoroma and lung cancer at a dose of 45 mg/kg when compared to the common injection formulation at 20 mg/kg and the albumin-bound paclitaxel nanoparticle injection formulation at 45 mg/kg, and the tumor growth is significantly slower than the common injection formulation and the albumin-bound paclitaxel nanoparticle injection formulation.

In the following section, the present invention will be further explained referring to description of figures and examples, however, technicians in this field should be aware of that the present invention is not limited to these examples and preparing procedure used. What's more, technicians in this field could perform equivalent substitution, combination, improvement or modification, however, all of these have been included in what is claimed the present invention.

### DESCRIPTION OF FIGURES

FIG. 1: HPLC examination graph of submicron emulsion in experimental example 2 (figure A is paclitaxel control; figure B is blank emulsion; figure C is submicron emulsion 14 preliminary; figure D is submicron emulsion 14 stored at 4° C. up to 12 months; figure E is submicron emulsion 31 stored at 4° C. up to 12 months. In which, peak 1 is paclitaxel, peak 2˜3 are impurities).

FIG. 2: experimental example 6, the inhibitory effect of different formulations on MDA-MB-231 tumor.

FIG. 3: experimental example 6, change of tumor bearing rate in MDA-MB-231 mouse model after giving different formulations.

FIG. 4: Example 6, the inhibitory effect of different formulations on the weight of nude mouse with MDA-MB-231 tumor.

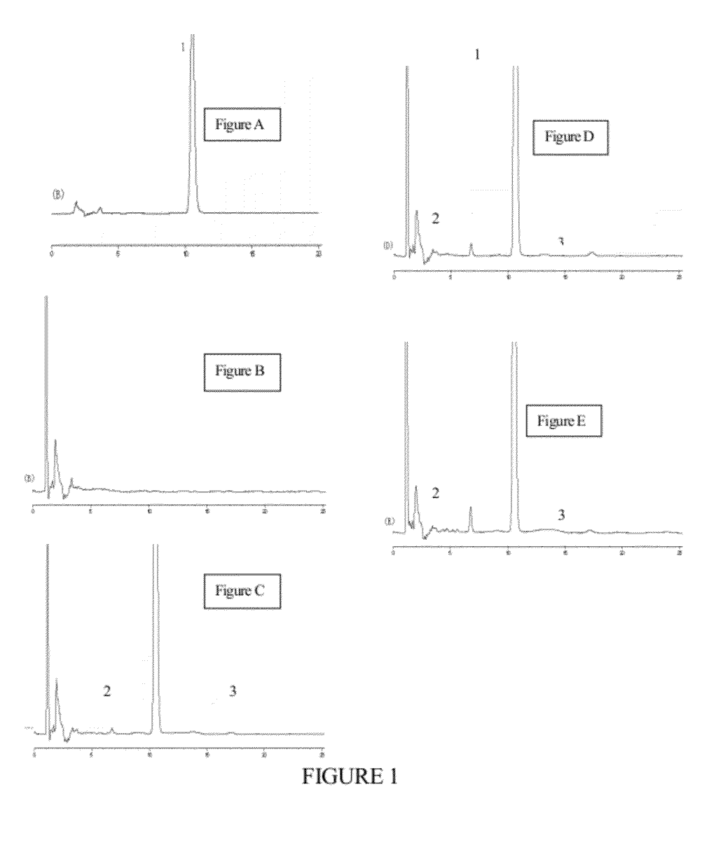

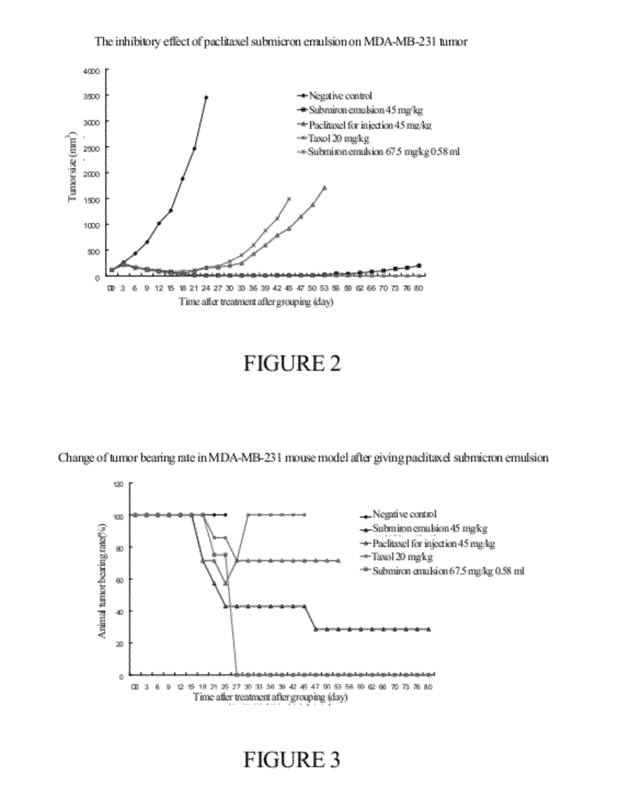

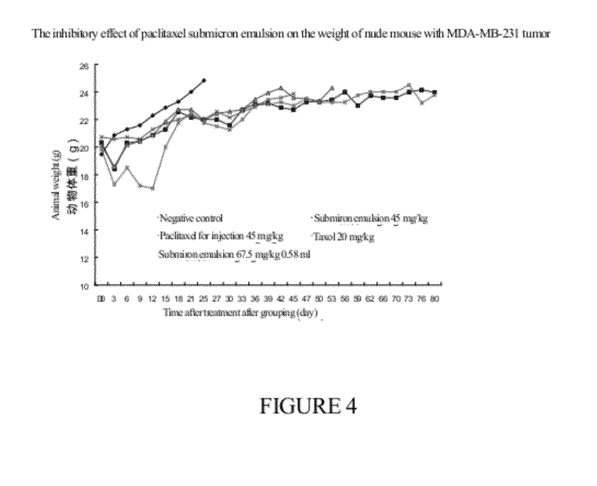

### EXAMPLES

### Example 1

### Paclitaxel/Steroid Complex

Test Complex 1˜Complex 6: take cholesterol, 7-hydrogenated cholesterol and Ergosterol as the lipid material according to the technical requirements of the invention patent, prepare two paclitaxel/cholesterol complexes (at a molar ratio of 1:1 and 1:2), two paclitaxel/7-hydrogenated cholesterol complexes (at a molar ratio of 1:1 and 1:4), and two paclitaxel/Ergosterol complexes (at a molar ratio of 1:1 and 1:4). Preparation method: dissolve paclitaxel and steroid in a flask, by adding 2000 ml acetone, with constant stirring gently at 40° C. for 1 hour, combine the washing to a rotary evaporator, remove from solvent, decompressed and dried in vacuum at 40° C. for 24 hours.

Reference Complex 1˜Reference Complex 4: Using phospholipid and cholesterol as the lipid material according to the technical requirements for complex of CN200810168212.5, prepare 2 Paclitaxel/phospholipid complexes (at a molar ratio of 1:6 and 1:10), 2 Paclitaxel/cholesterol complexes (at a molar ratio is 1:10 and 1:20). Its preparation method was the same as Test Complex 1˜Complex 6.

Composition and Preparation results of 6 Complexes and 4 Reference Complexes are provided below.

TABLE 1
Composition of Paclitaxel/steroid complex

| | | | Paclitaxel/ | |
|---|---|---|---|---|
| | | Lipid | Lipid material | |
| | Paclitaxel | material | Molar | Weight |
| Complex No./Lipid material | inventory | inventory | ratio | ratio |
| Test Complex 1/cholesterol | 8.0 g | 3.6 g | 1:1 | 1:0.45 |
| Test Complex 2/cholesterol | 8.0 g | 7.2 g | 1:2 | 1:0.90 |
| Test Complex 3/ | 3.0 g | 1.35 g | 1:1 | 1:0.45 |
| 7-hydrogenated cholesterol | | | | |
| Test Complex 4/ | 3.0 g | 5.406 g | 1:4 | 1:1.80 |
| 7-hydrogenated cholesterol | | | | |
| Test Complex 5/Ergosterol | 3.0 g | 1.40 g | 1:1 | 1:0.46 |
| Test Complex 6/Ergosterol | 3.0 g | 5.58 g | 1:4 | 1:1.86 |
| Reference Complex 1/ | 3.0 g | 16.65 g | 1:6 | 1:5.55 |
| phospholipid | | | | |
| Reference Complex 2/ | 3.0 g | 6.78 g | 1:10 | 1:9.23 |
| phospholipid | | | | |
| Reference Complex 3/ | 3.0 g | 13.5 g | 1:10 | 1:4.50 |
| cholesterol | | | | |
| Reference Complex 4/ | 3.0 g | 27.18 g | 1:20 | 1:9.06 |
| cholesterol | | | | |

### Example 2

### Paclitaxel Submicron-Emulsion Using Paclitaxel Cholesterol Complex as Intermediate Carrier

[Composition]

| | submicron | | submicron | | submicron | | submicron | |
|---|---|---|---|---|---|---|---|---|
| Component | emulsion 1 | | emulsion 2 | | emulsion 3 | | emulsion 4 | |
| Test Complex | 145 | mg | 290 | mg | 580 | mg | 160 | mg |
| 1* | | | | | | | | |
| Egg Yolk | 2 | g | 2.4 | g | 3 | g | 3 | g |
| Lecithin | | | | | | | | |
| Poloxamer | 1 | g | 2 | g | 4 | g | 6 | g |
| (188) | | | | | | | | |
| Glycerol | 5 | g | 5 | g | 5 | g | 5 | g |
| Soybean oil | 40 | ml | 40 | ml | 50 | ml | 50 | ml |
| Water for | 200 | ml | 200 | ml | 200 | ml | 200 | ml |
| injection | | | | | | | | |
| added to | | | | | | | | |
| Volume dose | 200 | ml | 200 | ml | 200 | ml | 200 | ml |

*Test Complex 1 was the complex prepared by Example 1, having a weight ratio of Paclitaxel/cholesterol of 1:0.45.

[Preparation Method]
Disperse the measured Egg Yolk Lecithin, poloxamer (188) and glycerol with 130-140 ml water for injection in the blender, stirring to form a homogeneous water phase, heat to 40° C., keep warm;
Heat the measured soybean oil to 40° C., weigh the Paclitaxel cholesterol Test Complex 1 prepared by Example 1, dissolve the complex in soybean oil, stir to form a homogeneous oil phase in the blender;
The water phase was added slowly to the oil phase under stirring conditions, at the rotation speed of 10000/min for emulsifying of 5 min, and transferred onto a High Pressure Homogenizer and homogenize for 6 times, collect the emulsion, adjust PH value to 4.0±0.5 with 0.1 mol/L HCl, and add water to 200 ml, and mix well, sterilization for 30 min at 115° after separate-loading.

Contents of Submicron emulsion 1—Submicron emulsion 4: the emulsifying agent (Egg Yolk Lecithin) constituted 1.0% (g/ml),1.2% (g/ml),1.5% (g/ml) and 1.5% (g/ml) of the submicron-emulsion respectively, and the co-emulsifier agent poloxamer (188) constituted 0.5% (g/ml),1.0% (g/ml),2.0% (g/ml) and 3.0% (g/ml), and the drug loading rate of paclitaxel was 0.5 mg/ml, 1.0 mg/ml, 2.0 mg/ml, 4.0 mg/ml respectively. The average particle diameter of 4 groups of emulsions was 225 nm, 233 nm, 245 nm, 230 nm, respectively, determined by laser particle sizer.

### Example 3

### Paclitaxel Submicron-Emulsion Using Paclitaxel Cholesterol Complex as Intermediate Carrier

Composition]

| | submicron | | submicron | | submicron | | submicron | |
|---|---|---|---|---|---|---|---|---|
| Component | emulsion 5 | | emulsion 6 | | emulsion 7 | | emulsion 8 | |
| Test Complex | 190 | mg | 380 | mg | 760 | mg | 1520 | mg |
| 2* | | | | | | | | |
| Egg Yolk | 2 | g | 2.4 | g | 3 | g | 3 | g |
| Lecithin | | | | | | | | |
| Poloxamer | 2.4 | g | 4 | g | 4 | g | 6 | g |
| (188) | | | | | | | | |
| Glycerol | 5 | g | 5 | g | 5 | g | 5 | g |
| Vitamin E | / | | / | | / | | 40 | mg |
| Soybean oil | 40 | ml | 40 | Ml | 50 | ml | 50 | ml |
| Water for | 200 | ml | 200 | ml | 200 | ml | 200 | ml |
| injection | | | | | | | | |
| added to | | | | | | | | |
| Volume dose | 200 | ml | 200 | ml | 200 | ml | 200 | ml |

*Test Complex 2 was the complex prepared by Example 1, having a weight ratio of Paclitaxel/cholesterol of 1:0.90.

[Preparation Method]
Weigh Egg Yolk Lecithin, poloxamer (188) and glycerol, dissolve in 130-140 ml water for injection, stir to form a homogeneous water phase in the blender, and heat to 40-80° C., keep warm;
Heat the measured soybean oil to 80° C., weigh Complex 2 Egg Yolk Lecithin and Vitamin E, dissolve in soybean oil, stir to form a homogeneous oil phase in the blender;
The water phase was added slowly to the oil phase under stirring conditions, at the rotation speed of 10000/min for emulsifying of 5 min, and transferred onto a High Pressure Homogenizer and homogenize for 6 times, collect the emulsion, adjust the PH value to 5.5±0.5 with 0.1 mol/L HCl, add water to 200 ml, mix well, and sterilize for 30 min at 115° C. after separate-loading.

Contents of Submicron emulsion 5-Submicron emulsion 8: the emulsifying agent (Egg Yolk Lecithin) constituted 1.0% (g/ml),1.2% (g/ml),1.5% (g/ml) and 1.5% (g/ml) of the submicron-emulsion respectively, and the co-emulsifier agent poloxamer (188) constituted 1.2% (g/ml, 2.0% (g/ml),2.0% (g/ml) and 3.0% (g/ml), and the drug loading rate of paclitaxel was 0.5 mg/ml, 1.0 mg/ml, 2.0 mg/ml, and 4.0 mg/ml respectively. The average particle diameter of 4 groups of emulsions is 246 nm, 262 nm, 231 m and 242 nm, respectively determined by laser particle sizer.

### Example 4

### Paclitaxel Submicron-Emulsion Using Paclitaxel Cholesterol Complex as Intermediate Carrier

[Composition]

| | | | submicron | submicron |
|---|---|---|---|---|
| | submicron | submicron | emulsion | emulsion |
| Component | emulsion 9 | emulsion 10 | 11 | 12 |
| Test Complex 1* | 145 mg | 290 mg | 580 mg | 1450 mg |
| soyabean lecithin | 2.4 g | 2.4 g | 2.4 g | 3.0 g |
| poloxamer(188) | 4 g | 4 g | 4 g | 4 g |
| Glycerol | 5 g | 5 g | 5 g | 5 g |
| Vitamin E | / | / | / | 40 mg |
| MCO | 40 ml | 40 ml | 50 ml | 50 ml |
| Water for injection | 200 ml | 200 ml | 200 ml | 200 ml |
| added to | | | | |
| Volume dose | 200 ml | 200 ml | 200 ml | 200 ml |

*Test Complex 1 was the complex prepared by Example 1, having a weight ratio of paclitaxel/cholesterol of 1:0.45.
MCO: medium chain oil

[Preparation Method]

It was the same as Example 3. The pH value of submicron emulsion was adjusted to 5.0±0.5.

Contents of emulsion 9-Submicron emulsion 12: the emulsifying agent (soyabean lecithin) constituted 1.2% (g/ml), 1.2% (g/ml), 1.2% (g/ml) and 1.5% (g/ml) of the submicron-emulsion respectively, and the co-emulsifier agent poloxamer (188) constituted 2.0% (g/ml) of the submicron-emulsion, and the drug loading rate of paclitaxel was 0.5 mg/ml, 1.0 mg/ml, 2.0 mg/ml, and 5.0 mg/ml, respectively. The average particle diameter of 4 groups of emulsions was 165 nm, 153 nm, 127 nm, and 138 nm, respectively, determined by laser particle sizer.

### Example 5

### Paclitaxel Submicron-Emulsion Using Paclitaxel Cholesterol Complex as Intermediate Carrier

[Composition]

| | | | submicron | submicron |
|---|---|---|---|---|
| | submicron | submicron | emulsion | emulsion |
| Component | emulsion 13 | emulsion 14 | 15 | 16 |
| Test Complex 1* | 290 mg | 435 mg | 580 mg | 1450 mg |
| soyabean lecithin | 2.4 g | 2.4 g | 2.4 g | 4.0 g |
| poloxamer(188) | 3 g | 3 g | 4 g | 4 g |
| Glycerol | 5 g | 5 g | 5 g | 5 g |
| Oil mixture** | 40 ml | 40 ml | 40 ml | 50 ml |
| Water for injection | 200 ml | 200 ml | 200 ml | 200 ml |
| added to | | | | |
| Volume dose | 200 ml | 200 ml | 200 ml | 200 ml |

*Test Complex 1 was the complex prepared by Example 1, having a weight ratio of Paclitaxel/cholesterol of 1:0.45.
**Oil mixture was the mixture of soybean oil/MCO (volume ratio 1:1).

[Preparation Method]

It was the same as Example 3. The pH value of Submicron emulsion is adjusted to 4.5±0.5.

Contents of Submicron emulsion13-Submicron emulsion16: the emulsifying agent (soyabean lecithin) constituted 1.2% (g/ml), 1.2% (g/ml), 1.2% (g/ml) and 2.0% (g/ml) of the submicron-emulsion, respectively, and the co-emulsifier agent poloxamer (188) constituted 1.5% (g/ml), 1.5% (g/ml), 2.0% (g/ml) and 2.0% (g/ml), respectively, and the drug loading rate of paclitaxel was 1.0 mg/ml, 1.5 mg/ml, 2.0 mg/ml and 5.0 mg/ml, respectively. The average particle diameter of 4 groups of emulsion was 145 nm, 138 nm, 133 nm, and 146 nm, respectively, determined by laser particle size.

### Example 6

### Paclitaxel Submicron-Emulsion Using Paclitaxel Cholesterol Complex as Intermediate Carrier

[Composition]

| | | | submicron | submicron |
|---|---|---|---|---|
| | submicron | submicron | emulsion | emulsion |
| Component | emulsion 17 | emulsion 18 | 19 | 20 |
| Test Complex 2* | 190 mg | 380 mg | 760 mg | 1520 mg |
| Egg Yolk Lecithin | 3.0 g | 3.0 g | 4.0 g | 6.0 g |
| poloxamer(188) | 4 g | 4 g | 6 g | 6 g |
| Glycerol | 5 g | 5 g | 5 g | 5 g |
| Oil mixture** | 30 ml | 40 ml | 50 ml | 60 ml |
| Water for injection | 200 ml | 200 ml | 200 ml | 200 ml |
| added to | | | | |
| Volume dose | 200 ml | 200 ml | 200 ml | 200 ml |

*Test Complex 2 was the complex prepared by Example 1, having a weight ratio of Paclitaxel/cholesterol of 1:0.90.
**Oil mixture was the mixture of soybean oil/MCO (volume ratio 1:1).

[Preparation Method]

It was the same as Example 2. The pH value of Submicron emulsion is adjusted to 5.5±0.5.

Contents of Submicron emulsion17-Submicron emulsion20: the emulsifying agent (Egg Yolk Lecithin) was 1.5% (g/ml),1.5% (g/ml), 2.0% (g/ml) and 3.0% (g/ml) of the submicron-emulsion respectively, and the co-emulsifier agent poloxamer (188) was 2.0% (g/ml), 2.0% (g/ml), 3.0% (g/ml) and 3.0% (g/ml), respectively, and the drug loading rate of paclitaxel was 0.5 mg/ml), 1.0 mg/ml), 2.0 mg/ml and 5.0 mg/ml, respectively. The average particle diameter of 4 groups of emulsions was 255 nm, 263 nm, 285 nm, and 232 nm, respectively, determined by laser particle sizer.

### Example 7

### Paclitaxel Submicron-Emulsion Using Paclitaxel Cholesterol Complex as Intermediate Carrier

[Composition]

| | | | submicron | submicron |
|---|---|---|---|---|
| | submicron | submicron | emulsion | emulsion |
| Component | emulsion 21 | emulsion 22 | 23 | 24 |
| Test Complex 1* | 145 mg | 290 mg | 580 mg | 1160 mg |
| Fatty glyceride | 3.0 g | 4.0 g | / | / |
| Polyoxyethylene- | / | / | 4.0 g | 6.0 g |
| Sorbitan Fatty | | | | |
| Acid Ester | | | | |
| poloxamer(188) | 3 g | 4 g | 4 g | 6 g |
| Glycerol | 5 g | 5 g | 5 g | 5 g |
| Oleic acid | 0.2 g | 0.2 g | 0.2 g | 0.2 g |
| Oil mixture** | 30 ml | 40 ml | 50 ml | 50 ml |
| Water for injection | 200 ml | 200 ml | 200 ml | 200 ml |
| added to | | | | |
| Volume dose | 200 ml | 200 ml | 200 ml | 200 ml |

*Test Complex 1 was the complex prepared by Example 1, having a weight ratio of Paclitaxel/cholesterol of 1:0.45.
**Oil mixture was the mixture of soybean oil/MCO (volume ratio 1:1).

[Preparation Method]

It was the same as Example 3.

In Submicron emulsion 21 and 22, the content of the fatty glyceride emulsifying agent was 1.5% (g/ml) and 2.0% (g/ml) of the submicron-emulsion, and in Submicron emulsion 23 and 24, the content of submicron-emulsion Polyoxyethylene Sorbitan Fatty Acid Ester is 2.0% (g/ml) and 3.0% (g/ml) respectively. The Ccontent of co-emulsifier agent poloxamer (188) was 1.5% (g/ml), 2.0% (g/ml), 2.0% (g/ml) and 3.0% (g/ml) respectively, and the drug loading rate of paclitaxel was 0.5 mg/ml, 1.0 mg/ml, 2.0 mg/ml and 4.0 mg/ml respectively. The average particle diameter of 4 groups of emulsions was 145 nm, 133 nm, 126 nm, 158 nm respectively determined by laser particle sizer.

### Example 8

### Submicron Emulsion Using 7-Hydrogenated Cholesterol or Ergosterol Complex as Intermediate Carrier

[Composition]

| | | | submicron | submicron |
|---|---|---|---|---|
| | submicron | submicron | emulsion | emulsion |
| Component | emulsion 25 | emulsion 26 | 27 | 28 |
| Complex 3* | 290 mg | / | / | / |
| Complex 4* | / | 5604 mg | / | / |
| Complex 5* | / | / | 292 mg | / |
| Complex 6* | / | / | / | 5720 mg |
| soyabean lecithin | 2.4 g | 2.4 g | 2.4 g | 4.0 g |
| poloxamer(188) | 3 g | 3 g | 4 g | 4 g |
| Glycerol | 5 g | 5 g | 5 g | 5 g |
| Oil mixture** | 40 ml | 40 ml | 40 ml | 50 ml |
| Water for injection | 200 ml | 200 ml | 200 ml | 200 ml |
| added to | | | | |
| Volume dose | 200 ml | 200 ml | 200 ml | 200 ml |

*Complex 3~Complex 6 were the Paclitaxel/7-hydrogenated cholesterol complex and the Paclitaxel/Ergosterol complex prepared by Example 1
**Oil mixture was the mixture of soybean oil/MCO (volume ratio 1:1).

[Preparation Method]

The same as Example 5.

In submicron emulsion25-Submicron emulsion28, the drug loading rate of paclitaxel was 1.0 mg/ml. The average particle diameter of 4 groups of emulsions was 143 nm, 138 nm, 141 nm, 132 nm, respectively, determined by laser particle sizer.

### Example 9

### Dry Emulsion Using Paclitaxel Cholesterol Complex as Intermediate Carrier

Dissolve the 28 groups of Submicron emulsions 50 ml each prepared by Example 2-Example 8 in 3% (w/v) mannitol with constant stirring, and filter with 0.2 μm millipore filter, and dried at a lower temperature.

### Test Example

### Test Example 1

### Steroid-Sparing Effect on Entrapment Efficiency of Submicron-Emulsion

Determination Method of Entrapment Efficiency

Determination of Total Drug Content of a Submicron-Emulsion:

Measure accurately 10 mL of an emulsion, dilute by adding anhydrous alcohol in a 250 mL volumetric flask after demulsification, and mix well to obtain a test solution. Measure accurately some reference Paclitaxel in a flask by adding dehydrated alcohol and dilute to 40 μg/mL as a reference solution. Weigh accurately 20 μL of the test solution and the reference solution each, and inject in a Kromasil-C18 (300 mm×4.6 mm, 5 μm) column for an HPLC analysis. The mobile phase consisted of acetonitrile-water (54:46), the flow rate: 1.0 mL/min, the detection wavelength: 230 nm, the column temperature: room temperature, and sample volume: 10 μL. Record the chromatography chart and calculate the peak area and concentration of Paclitaxel and total drug content in the emulsion by the external standard method, record as W Total.

Determination of Total Drug Content in the Water Phase:

Centrifuge 10 mL of an emulsion on 40,000 r/min at 16° C. for 4.0 h, to obtain separate layers: from the top, the oil layer, the emulsifying agent layer and the aqueous solution layer successively. Record the volume of the aqueous solution layer, carefully remove the oil layer and the emulsifying agent layer, carefully take the clarified aqueous solution layer in the bottom, and filter with 0.22 μm millipore filter, the resulting filtrate was directly sampled to determine the concentration by the HPLC method described above, and calculate the drug content according to the volume of the water phase, record as W water phase.

The total drug content of the W oil phase and oil-water surface:

W oil phase+oil-water surface=W Total-W water phase,

$$Calculation of entrapment efficiency \text{:} Entrapment efficiency == \frac{\begin{matrix}W oil phase + \\ oil \text{-} water surface\end{matrix}}{WTOTAL} \times 100 \% \frac{( W Total - water phase )}{WTOTAL} \times 100 \%$$

Determination of Entrapment Efficiency in Submicron-Emulsion 1˜Submicron-Emulsion 28

Take the 28 groups of submicron-emulsions prepared by Example 2-Example 7, calculate the entrapment efficiency as described above, and the results are shown in Table 2. The results indicate that if the content of steroid of the intermediate carrier was 0.09˜1.86 of Paclitaxel (take cholesterol as the lipid material, the content was 0.45˜0.90 of Paclitaxel), the entrapment efficiency of submicron-emulsions (with different drug loading rates) were above 90%; and when the drug loading rate was below 2 mg/ml, the entrapment efficiency reached more than 95%, and less drugs dissociated into the water phase.

TABLE 2
Determination Results of Entrapment efficiency in 28 groups of
submicron-emulsion

| | Drug loading | Entrapment |
|---|---|---|
| Sample number | rate (mg/ml) | efficiency |
| Submicron emulsion1 | 0.5 | 96.2% |
| Submicron emulsion2 | 1.0 | 95.1% |
| Submicron emulsion3 | 2.0 | 95.7% |
| Submicron emulsion4 | 3.0 | 93.5% |
| Submicron emulsion5 | 0.5 | 97.7% |
| Submicron emulsion6 | 1.0 | 96.8% |
| Submicron emulsion7 | 2.0 | 96.7% |
| Submicron emulsion8 | 4.0 | 92.6% |
| Submicron emulsion9 | 0.5 | 98.2% |
| Submicron emulsion10 | 1.0 | 98.9% |
| Submicron emulsion11 | 2.0 | 97.6% |
| Submicron emulsion12 | 5.0 | 91.3% |
| Submicron emulsion13 | 1.0 | 98.8% |
| Submicron emulsion14 | 1.5 | 98.1% |
| Submicron emulsion15 | 2.0 | 97.5% |
| Submicron emulsion16 | 5.0 | 90.8% |
| Submicron emulsion17 | 0.5 | 96.5% |
| Submicron emulsion18 | 1.0 | 96.8% |
| Submicron emulsion19 | 2.0 | 97.2% |
| Submicron emulsion20 | 5.0 | 90.5% |
| Submicron emulsion21 | 0.5 | 98.6% |
| Submicron emulsion22 | 1.0 | 97.1% |
| Submicron emulsion23 | 2.0 | 97.2% |
| Submicron emulsion24 | 4.0 | 93.3% |
| Submicron emulsion25 | 1.0 | 98.9% |
| Submicron emulsion26 | 1.0 | 97.1% |
| Submicron emulsion27 | 1.0 | 98.4% |
| Submicron emulsion28 | 1.0 | 98.2% |

Preparation of Reference Submicron-Emulsion & Determination of Entrapment Efficiency

Take Reference Complex 1-Reference Complex 4 prepared by Example 1, prepare the submicron-emulsion 29˜submicron-emulsion 32 as follows, and the drug loading rate was 0.5, 1.0, 1.0 and 2.0 mg/ml respectively for comparison investigation.

Prescription, Preparation method and measured entrapment efficiencies are as follows:

[Composition]

| | | submicron | submicron | submicron |
|---|---|---|---|---|
| | submicron | emulsion | emulsion | emulsion |
| Component | emulsion 29 | 30 | 31 | 32 |
| Reference Complex 1* | 655 mg | / | / | / |
| Reference Complex 2* | / | 1023 mg | / | / |
| Reference Complex 3* | / | / | 1100 mg | / |
| Reference Complex 4* | / | / | / | 4024 mg |
| Egg Yolk Lecithin | 3 g | 3 g | 3 g | 3 g |
| Poloxamer188 | 3 g | 3 g | 3 g | 3 g |
| Glycerol | 5 g | 5 g | 5 g | 5 g |
| Soybean oil | 40 ml | 40 ml | 50 ml | 50 ml |
| Water for injection | 200 ml | 200 ml | 200 ml | 200 ml |
| added to | | | | |
| Volume dose | 200 ml | 200 ml | 200 ml | 200 ml |

*Reference Complex 1,2 were the two groups of Reference Complexes prepared by Example 1, with weight ratios of Paclitaxel/phospholipid being 1:5.55 and 1:9.23 respectively.
*Reference Complex 3,4 were the two groups of Reference Complexes prepared by Example 1, with weight ratios of cholesterol Paclitaxel/phospholipid being 1:4.50 and 1:9.06 respectively.

[Preparation Method]

Heat the measured soybean oil to 40° C.-80° C., add Paclitaxel cholesterol Test Complex 1 prepared by Example 1 into the soybean oil, stir to form a homogeneous oil phase in the blender;

Weigh Egg Yolk Lecithin, poloxamer (188) and glycerol, dissolve in 130-140 ml water for injection, stir to form a homogeneous water phase in the blender, and heat to 40-80° C., keep warm;

The water phase was added slowly to the oil phase under stirring conditions, at the rotation speed of 10000-20000/min for emulsification for 5-10 min, and the resulting preliminary emulsion was transferred onto a High Pressure Homogenizer and homogenized for 6 times, collect the emulsion, adjust the pH value to 4.5±0.5 with 0.1 mol/L HCl, and add water to 200 ml, and mix well, sterilize for 30 min at 115° C. after separate-loading.

[Determination of Entrapment Efficiency]

Take submicron-emulsion 29 to submicron-emulsion 32, determine the entrapment efficiency by the method mentioned above. The entrapment efficiencies were 65.7%˜84.5%. Please see the Table 3 below.

TABLE 3
Determination Result of Entrapment efficiency in Reference submicron-
emulsion (Submicron emulsion 29~Submicron emulsion 32)

| Sample of reference Submicron | Results of |
|---|---|
| emulsion | Entrapment efficiency |
| Submicron emulsion 29 | 75.1% |
| Submicron emulsion 30 | 65.7% |
| Submicron emulsion 31 | 84.5% |
| Submicron emulsion 32 | 83.2% |

### Test Example 2

### Stability Study of Submicron-Emulsions

Take the 28 groups of submicron-emulsions prepared by Example 2-Example 8 (marked as Emulsion1˜28 in the following tables) and the 4 groups of reference submicron-emulsions prepared by Test example 1 (record as Emulsion29˜Emulsion32 in the following tables), store these at 4° for 12 months respectively, samples were measured at 0, 6 and 12 month. The differences in appearance, particle diameter, purity and impurity were investigated by the following method.

Character:

visual method, record the color of submicron-emulsions, record whether there were oil droplets or phase separation on the surface.

Particle Diameter:

Determine the particle diameter of the submicron-emulsions by MASTER SIZER 2000 laser particle sizer (MALVERN).

Purity and Related Substances:

Measure accurately a defined amount of a Paclitaxel submicron-emulsion and add anhydrous alcohol with a demulsification method, to prepare a test solution with a suitable concentration. Weigh accurately 20 μL of the test solution, inject in a chromatography column for an HPLC analysis—the column: Kromasil-C18 (300 mm×4.6 mm, 5 μm), the mobile phase consisted of acetonitrile-water (54:46), the flow rate: 1.0 mL/min, the detection wavelength: 230 nm, the column temperature: room temperature, record the chromatograph chart and calculate the total drug content of the emulsion according to the peak area by the external standard method, calculate impurity content by using the normalization method.

Result:

See the table below.

TABLE 4
Comparative result of stability among submicron-emulsion

| | The original | | | | | | | |
|---|---|---|---|---|---|---|---|---|
| Sample | (determined within a week after preparation) | | | | Keep for 12 months at 4° | | | |
| number/Drug | | particle | | | | Particle | | |
| loading rate | Appearance | diameter | Purity | Impurity | Uniform | diameter | Purity | Impurity |
| Emulsion1/0.5 mg/ml | Uniform | 225 nm | 100.2% | 0.33% | Uniform | 221 nm | 99.7% | 0.67% |
| Emulsion2/1.0 mg/ml | Uniform | 233 nm | 97.6% | 0.31% | Uniform | 245 nm | 97.3% | 0.62% |
| Emulsion3/2.0 mg/ml | Uniform | 245 nm | 98.5% | 0.31% | Uniform | 236 nm | 98.2% | 0.64% |
| Emulsion4/3.0 mg/ml | Uniform | 230 nm | 97.6% | 0.37% | Uniform | 237 nm | 97.0% | 0.93% |
| Emulsion5/0.5 mg/ml | Uniform | 246 nm | 99.5% | 0.35% | Uniform | 228 nm | 98.8% | 0.52% |
| Emulsion6/1.0 mg/ml | Uniform | 262 nm | 100.3% | 0.30% | Uniform | 255 nm | 99.5% | 0.61% |
| Emulsion7/2.0 mg/ml | Uniform | 231 nm | 98.1% | 0.36% | Uniform | 240 nm | 98.6% | 0.57% |
| Emulsion8/4.0 mg/ml | Uniform | 242 nm | 99.6% | 0.42% | Uniform | 251 nm | 97.3% | 1.01% |
| Emulsion9/0.5 mg/ml | Uniform | 165 nm | 98.8% | 0.35% | Uniform | 126 nm | 99.2% | 0.56% |
| Emulsion10/1.0 mg/ml | Uniform | 153 nm | 101.6% | 0.30% | Uniform | 133 nm | 100.7% | 0.52% |
| Emulsion11/ | Uniform | 127 nm | 102.2% | 0.32% | Uniform | 131 nm | 100.9% | 0.55% |
| 2.0 mg/ml | | | | | | | | |
| Emulsion12/5.0 mg/ml | Uniform | 138 nm | 99.8% | 0.45% | Uniform | 155 nm | 99.4% | 1.24% |
| Emulsion13/1.0 mg/ml | Uniform | 145 nm | 97.4% | 0.31% | Uniform | 138 nm | 98.0% | 0.57% |
| Emulsion14/1.5 mg/ml | Uniform | 138 nm | 100.3% | 0.35% | Uniform | 136 nm | 98.7% | 0.65% |
| Emulsion15/ | Uniform | 133 nm | 99.2% | 0.31% | Uniform | 137 nm | 98.5% | 0.63% |
| 2.0 mg/ml | | | | | | | | |
| Emulsion16/5.0 mg/ml | Uniform | 146 nm | 98.5% | 0.43% | Uniform | 173 nm | 97.2% | 1.68% |
| Emulsion17/ | Uniform | 255 nm | 97.8% | 0.32% | Uniform | 244 nm | 97.6% | 0.57% |
| 0.5 mg/ml | | | | | | | | |
| Emulsion18/1.0 mg/ml | Uniform | 263 nm | 99.2% | 0.36% | Uniform | 259 nm | 99.5% | 0.53% |
| Emulsion19/2.0 mg/ml | Uniform | 285 nm | 100.4% | 0.33% | Uniform | 272 nm | 98.8% | 0.61% |
| Emulsion20/5.0 mg/ml | Uniform | 232 nm | 101.4% | 0.48% | Uniform | 258 nm | 97.8% | 1.76% |
| Emulsion21/0.5 mg/ml | Uniform | 145 nm | 98.7% | 0.36% | Uniform | 136 nm | 99.1% | 0.59% |
| Emulsion22/ | Uniform | 133 nm | 98.2% | 0.32% | Uniform | 127 nm | 97.6% | 0.57% |
| 1.0 mg/ml | | | | | | | | |
| Emulsion23/2.0 mg/ml | Uniform | 126 nm | 101.1% | 0.38% | Uniform | 134 nm | 99.5% | 0.56% |
| Emulsion24/4.0 mg/ml | Uniform | 158 nm | 98.8% | 0.41% | Uniform | 163 nm | 97.9% | 1.25% |
| Emulsion25/1.0 mg/ml | Uniform | 143 nm | 99.5% | 0.33% | Uniform | 128 nm | 98.9% | 0.56% |
| Emulsion26/1.0 mg/ml | Uniform | 138 nm | 97.8% | 0.35% | Uniform | 132 nm | 97.4% | 0.58% |
| Emulsion27/1.0 mg/ml | Uniform | 141 nm | 99.4% | 0.30% | Uniform | 145 nm | 99.1% | 0.61% |
| Emulsion28/1.0 mg/ml | Uniform | 132 nm | 98.6% | 0.36% | Uniform | 126 nm | 98.2% | 0.54% |
| Emulsion29/0.5 mg/ml | Uniform | 253 nm | 97.5% | 0.92% | layered | 323 nm | 90.8% | 7.63% |
| | | | | | and | | | |
| | | | | | floating oil | | | |
| Emulsion30/1.0 mg/ml | Layered | 522 nm | 93.2% | 6.27% | oil-water | Untested | Untested | Untested |
| | | | | | separation | | | |
| Emulsion31/1.0 mg/ml | Uniform | 247 nm | 98.6% | 0.62% | Uniform | 263 nm | 94.7% | 3.58% |
| Emulsion32/2.0 mg/ml | Uniform | 266 nm | 98.9% | 0.78% | Slightly | 311 nm | 92.8% | 4.64% |
| | | | | | layered | | | |

Keep Submicron emulsion1˜Submicron emulsion28 prepared by the steroid complex as the intermediate carrier of the invention in the refrigerator (4° C.) for 12 months, and compare with the original emulsions prior to the storage. It was found that 1) the average particle diameter of emulsions with a drug loading rate of 5.0 mg/ml had a trend of ascend, but there was no layer, and no obvious changes occurred in appearance or purity, and impurity increased but did not exceed 2.0%; 2) for emulsions with a drug loading rate of 4.0 mg/ml, there was no layer, and no obvious changes occurred in particle size, appearance or purity, and impurity increased but did not exceed 1.3%; 3) for emulsions with a drug loading rate of 3.0 mg/ml, there was no layer, and no obvious changes occurred in particle size, appearance or purity, and impurity did not exceed 1.0%; 4) for emulsions with a drug loading rate of 2.0 mg/ml or below, there was no layer, and no obvious changes occurred in particle size, appearance or purity, impurity did not exceed 0.7%.

With the emulsion prepared by using the reference Paclitaxel/phospholipid complex as the intermediate carrier, a uniform emulsion (submicron-emulsion 29) was formed with a drug loading rate of 0.5 mg/ml; if stored for 6 months, there was no layer, and no obvious changes occurred in appearance or purity, impurity increased to 3.0%; but if stored for 12 months, the particle diameter increased significantly and impurity increased to above 7%, purity decreased, and layers appeared with floating oil; 2) when the drug loading rate rose to 1.0 mg/ml, it was impossible to form a uniform emulsion (submicron-emulsion 30), drug crystals and oil droplets were observed at the beginning.

With the emulsion prepared by the reference Paclitaxel/cholesterol complex as the intermediate carrier, 1) uniform emulsions (submicron-emulsion 31˜32) were formed when the drug loading rate was 1.0 mg/ml and 2.0 mg/ml, respectively; if stored for 6 months, there was no layer, and no obvious changes occurred in appearance or purity in either of the 2 groups with different drug loading rates, impurity increased but did not exceed 1.5%; if stored for 12 months, the particle diameters of the 2 groups of emulsions increased, purities declined, and impurities rose to 3.58% and 4.64%, respectively; and when the drug loading rate rose to 2.0 mg/ml, the emulsion was slightly layered.

### Test Example 3

### Sensitization Test on Paclitaxel Submicron-Emulsion

Test Drugs:

Tested drug solution: Submicron-emulsion 14 prepared by Example 5;

Reference tablet solution: the commercial paclitaxel injection “Rhodoxanthin” (5 ml: 30 mg) diluted with normal saline solution with a concentration of Paclitaxel 2 mg/ml before use;

Positive drug solution: 1.0% ovalbumin;

Blank emulsion solution: prepared based on the composition of submicron-emulsion 14 in Example 5 without Paclitaxel cholesterol complex;

Blank solvent: Mix the Cremophor EL and Dehydrated Alcohol at 1:1(v/v) (simulated composition of the commercial paclitaxel injection, and without Paclitaxel), diluted with normal saline by 3 times before use;

Laboratory Animal:

Guinea-pigs (300 g±20 g, bisexual each half)

Methods:

30 guinea-pigs (300 g±20 g) were randomly divided into 5 groups, 6 in each group, bisexual each half. The guinea-pigs were kept for 1 week before test to observe their activities. Individual groups of guinea-pigs were given an intraperitoneal injection of a test drug solution (submicron-emulsion group), a Reference tablet solution (Reference tablet group), a positive control solution (positive control group), Blank Emulsion (Blank Emulsion group), and a Blank solvent (Blank solvent group), respectively, at a dose of 0.3 ml each, on every other day for 3 times and were sensitized, the test solution group were of Paclitaxel at 2 mg/kg. Twelve (12) days after the last injection, the individual groups were given an intravenous injection of the Test drug solution, the Reference tablet solution, the Positive drug solution, the Blank Emulsion and the Blank solvent, respectively, at a dose of 1.0 ml each, for stimulation, the test solution group were dosed of Paclitaxel at 6 mg/kg. Symptoms of each group of guinea-pigs were observed after the intravenous injection, and the results are shown in the table below.

TABLE 5
Sensitization Study of Paclitaxel Submicron emulsion

| Groups | Symptoms | Results |
|---|---|---|
| 1) Reference tablet group | Scratching the nose, tremble, | Strongly |
| | bristling, tachypnea, gait | Positive |
| | disturbance, dyspnea, spasm, | |
| | rotation, myasthenia of limbs | |
| 2) Submicron-emulsion group | Normal | negative |
| 3) Blank solvent group | Gait disturbance, dyspnea, | Strongly |
| | spasm, rotation, myasthenia | Positive |
| | of limbs | |
| 4) Blank Emulsion group | Normal | negative |
| 5) Positive control group | Scratching the nose, | Strongly |
| | tachypnea, dejection, gait | Positive |
| | disturbance, wheeze, spasm, | |
| | rotation | |

The results show that, the commercial Paclitaxel injection group and the blank solvent group had a strong allergy effect, while the Paclitaxel submicron-emulsion and the blank emulsion group had no obvious allergy effect.

### Test Example 4

### Evaluate the Acute Toxicity of Paclitaxel Submicron-Emulsion

Test Samples:

Submicron emulsion solution: take Submicron-emulsion 14 prepared by Example 5; Reference tablet solution: the commercial Paclitaxel injection with Cremophor EL (5 ml:30 mg), diluted with normal saline at a concentration of Paclitaxel 2 mg/ml before use.

Laboratory Animal:

70 Kunming mice, ♀, 20±2 g (Institute of laboratory animal of Chinese academy medical sciences).

Methods:

70 Kunming mice were randomly divided into 7 groups, 10 in each group according to their weight, injection administrations are as follows:

Group 1: Reference tablet solution was injected to have an initial dose at 25 mg/kg;

Group 2: Reference tablet solution was injected to have an initial dose at 29 mg/kg;

Group 3: Reference tablet solution was injected to have an initial dose at 25 mg/kg every 4 days.

Group 4: Submicron emulsion solution was injected to have an initial dose at 29 mg/kg for 2 days in succession;

Group 5: Submicron emulsion solution was injected to have an initial dose at 29 mg/kg for 3 days in succession;

Group 6: Submicron emulsion solution was injected to have an initial dose at 25 mg/kg every 4 days;

Group 7: Submicron emulsion solution was injected to have an initial dose at 44 mg/kg.

The results of acute toxicity are shown in the following table, please see sketch2 for weight changing curve.

TABLE 6
Results of acute toxicity experience

| Drug | Symptoms | Results |
|---|---|---|
| 1) Reference tablet | Jumping for several times, | Group (1) 1 |
| (Group 1-Group 3) | dyspnea or apnea, fatigue, | mouse died. |
| | eye closure, straddle fixedly, | Group (2) 1 |
| | tic and tremble (some of them). | mouse died. |
| | It was remitted in 5~20 min, | Group (3) 2 |
| | but there were still symptoms of | mice died. |
| | astasia and gait disturbance and | |
| | lasted for several hours. Weight | |
| | loss and turned darker some | |
| | days after injection. | |
| 2) Submicron- | No obvious immediate symptoms. | No death |
| emulsion | Weight loss some days after | in each group. |
| (Group 4-Group 7) | injection, significantly related | |
| | to doses. Colors of the mice | |
| | with high dose turned darker. | |

The results of Group 1, Group 2 and Group 3 show that, acute toxicity reactions appeared after injection of Reference tablet, 1 mouse died each in Group 1 and Group 2, 2 mice died in Group 3. No obvious immediate symptoms and death were observed in Group 4-Group 7.

Group 1 and Group 7 were injected at a single dose, but the dose in Group 7 is 1.765 times of Group 1, and toxicity of extraction in Group 7 is lower than Group 1, which indicates that the submicron emulsion was associated with lower toxicity and more tolerated dose than the Reference tablet (the commercial injection form).

### Test Example 5

### Maximum Tolerated Dose (MTD) Test of Paclitaxel Submicron-Emulsion

Laboratory Animal:

nude mice

Test Sample:

Commercial injection with Cremophor EL, divided into 3 groups at a dose of 20, 30 and 45 mg/kg respectively;

Submicron-emulsion 14 prepared by Example 5, divided into 4 groups at a doss of 30, 45, 67.5 and 101.25 mg/kg respectively;

Methods:

Intravenous injection at an initial dose every 4 days and for 3 times. Observe obvious immediate symptoms and death from pre-injection to a week post-injection, record the dose (without death) as the maximum tolerated dose (MTD). The results are shown below.

TABLE 7
MTD Results of nude mouse

| | | Numbers of | | |
|---|---|---|---|---|
| Test solution | Dose(mg/kg) | Animal | Deaths | MTD |
| Ordinary injection | 20 | 10 | 0 | 20 mg/kg |
| | 30 | 10 | 1 | |
| | 45 | 5 | 3 | |
| Submicron-emulsion | 30 | 10 | 0 | 45 mg/kg |
| | 45 | 10 | 0 | |
| | 67.5 | 8 | 1 | |
| | 101.25 | 8 | 2 | |

MTD of Ordinary injection was 20 mg/kg, and MTD of submicron-emulsion was 45 mg/kg. Inc contrast, MTD of Submicron emulsion rose to 2.25 times, which was similar to Abraxane (Protein-bound paclitaxel) reported in the literature.

### Test Example 6

### The Inhibition of Tumor of Paclitaxel Submicron-Emulsion

Purpose:

Observe the inhibition of Paclitaxel submicron-emulsion on human breast carcinoma MDA-MB-23 xenografts in nude mice at the maximum tolerated dose, then compare with Abraxane (Protein-bound paclitaxel) and a commercial injection (Paclitaxel).

Test Samples:

1) Submicron-emulsion 14 prepared by Example 5;

2) Commercial Paclitaxel injection (Paclitaxel, Beijing Union pharmaceutical factory), batch number 100102, 30 mg/5 ml;

3) Abraxane (Protein-bound paclitaxel) (Display as Abraxane), product of American Pharmaceutical Partners, batch number 205133, 100 mg/1 g;

Groups:

Negative control group, Paclitaxel injection group, Abraxane group, and Paclitaxel Submicron emulsion group

Paclitaxel injection group, at a dose of 20 mg/kg/once,

Paclitaxel submicron-emulsion group, at a dose of 45 mg/kg/once and 67.5 mg/kg/once,

Abraxane group, at a dose of 45 mg/kg/once.

Tumor Model:

Human breast carcinoma MDA-MB-23 xenografts in nude mice,

Tumor-bearing mouse is from crown Bioscience Inc. (Beijing)

Experiment Process and Observation Methods:

The healthy nude mice with MDA-MB-23 xenografts were sacrificed by cervical dislocation. Tissues of tumor were extracted in aseptic conditions, choose some good tissues and cut into blocks dir.2-3 mm with the scalpel, inoculated subcutaneously into the nude mice. And tumor grew naturally after inoculation.

Tumor-bearing mice were divided by tumor volume, 7 in each group when the tumors grew to 110˜120 mm³. Other groups of mice were given tail vein injection once every 4 days except Negative control group. (Paclitaxel Submicron emulsion (67.5 mg/kg) group were given intravenous injection half of volume dose and another half in 1-2 h later). Each group of mice were injected with intermittent administration for 3 times.

Record the first dose day as (D0), and detect changes of the tumors and weight of mice every 3 days while carrying out the experiment.

The 25^{th} day (D25), after 3 times of injection, tumors growth were inhibited, when test values of Tumor volumes were equal to or less than starting injection, mice in Negative control group with larger tumors were sacrificed and tumor growth of other groups remains to be observed.

The 45^{th} day (D45), the group of mice with Paclitaxel injection 20 mg/kg were sacrificed on D45 for the average Tumor volume was close to 1500 mm³.

The 53^{rd} day (D53), the group of mice with Abraxane injection 45 mg/kg were sacrificed on D53 for the average Tumor volume was up to 1700 mm³.

The 80^{th} day (D80), By the time you finish testing, the average Tumor volume of the mice with Paclitaxel submicron-emulsion injection 45 mg/kg is 198 mm³ in size, and in 67.5 mg/kg group the tumor disappeared, and there was no recurrence.

Conclusion: In conclusion, on a condition of MTD, anti-tumor rates of the 3 (commercial Paclitaxel Ordinary injection20 mg/kg, Paclitaxel submicron-emulsion 45 mg/kg and Abraxane 45 mg/kg) were above 98%, but the inhibitory effect of the 3 groups showed significant difference.

On the 25^{th} day, the relative Tumor volume of Paclitaxel Ordinary injection group and Abraxane group is 1.41 and 0.92 respectively, and Tumor in Paclitaxel submicron-emulsion group almost disappeared, and RTV was just 0.06.

On the 45^{th} day, Tumor in ordinary injection group grew fast, RTV was up to 14.3(were sacrificed); Tumor in Abraxane group grew slower than ordinary injection group, but RTV was up to 6.5; and there was almost no increase of Tumor volume in Paclitaxel submicron-emulsion group and RTV is 0.11.

On the 53^{rd} day, RTV of Abraxane group was reached to 12.5 ((were sacrificed), while in Paclitaxel submicron-emulsion group was 0.26.

On the 80^{th} day, RTV of Paclitaxel submicron-emulsion group was 1.83. And the mice in this group were sacrificed for slow growth of Tumor and finish testing.

TABLE 8
Tumor volume and Tumor condition on the 25^{th} day (D25)

| | | | | | Tumor condition | |
|---|---|---|---|---|---|---|
| | Tumor volume (mm³) | | | T/C | tumor/Mouse | |
| Groups | Primary D25 | | RTV | (%) | Tumor rate | |
| Negative control | 110 ± 62.4 | | 37.90 ± 17.32 | | 7/7 | 100% |
| group | 3452 ± 1080.8 | | | | | |
| Ordinary injection | 119 ± 55.7 | 165 ± 192.2 | 1.41 ± 1.170 | 3.72 | 7/7 | 100% |
| (Paclitaxel) | | | | | | |
| 20 mg/kg | | | | | | |
| BSANP for | 114 ± 32.3 | 158 ± 178.4 | 0.92 ± 1.102 | 2.42 | 5/7 | 71.4% |
| injection 45 mg/kg | | | | | | |
| Paclitaxel | 116 ± 61.0 | 8 ± 13.8 | 0.06 ± 0.074 | 0.15 | 5/7 | 71.4% |
| submicron-emulsion | 1122 ± 60.1 | 6 ± 4.8 | 0.08 ± 0.063 | 0.21 | 4/7-3* | 100% |
| 45 mg/kg | | | | | | |
| 67.5 mg/kg | | | | | | |

*Note: there are 3 mice died in this group during administration, and left 4. Following is the same.

TABLE 9
Tumor volume and Tumor condition on the 45^{th} day (D45)

| | | | | Tumor condition | |
|---|---|---|---|---|---|
| | Tumor volume (mm³) | | | tumor/Mouse | |
| Group | Primary D45 | | RTV | Tumor rate | |
| Ordinary injection | 119 ± 55.7 | | 14.34 ± 12.26 | 7/7 | 100% |
| (Paclitaxel) 20 mg/kg | 1491 ± 1305.3 | | | | |
| BSANP for | 114 ± 32.3 | 917 ± 873.4 | 6.53 ± 5.730 | 5/7 | 71.4% |
| injection | | | | | |
| 45 mg/kg | | | | | |
| Paclitaxel | 116 ± 61.0 | 12 ± 14.2 | 0.11 ± 0.154 | 3/7 | 42.9% |
| submicron-emulsion | 1122 ± 60.1 | 0 | 0 | 0/7-3* | 0 |
| 45 mg/kg | | | | | |
| 67.5 mg/kg | | | | | |

TABLE 10
Tumor volume and Tumor condition on the 53rd day (D53)

| | | | | Tumor condition | |
|---|---|---|---|---|---|
| | Tumor volume (mm³) | | | tumor/Mouse-borne | |
| Group | Primary D45 | | RTV | Tumor rate | |
| BSANP for Injection | 114 ± 32.3 | | 12.51 ± 10.745 | 5/7 | 71.4% |
| 45 mg/kg | 1706 ± 1499.4 | | | | |
| Paclitaxel | 116 ± 61.0 | 23 ± 43.6 | 0.26 ± 0.582 | 2/7 | 28.6% |
| submicron-emulsion | 1122 ± 60.1 | 0 | 0 | 0/7-3* | 0 |
| 45 mg/kg | | | | | |
| 67.5 mg/kg | | | | | |

TABLE 11
Tumor volume and Tumor condition on the 80^{th} day (D80)

| | | | Tumor | |
|---|---|---|---|---|
| | | | condition | |
| | Tumor volume | | tumor/ | |
| | (mm³) | | Mouse-borne | |
| Group | Primary D80 | RTV | Tumor rate | |
| Paclitaxel Submicron- | 116 ± 61.0 | 1.83 ± 3.624 | 2/7 | 28.6% |
| emulsion Group | 198 ± 317.1 | 0 | 0/7-3 | 0 |

| | | |
|---|---|---|
| 45 mg/kg | 1122 ± 60.1 | 0 |
| 67.5 mg/kg | | |

1. A paclitaxel submicron emulsion, which comprises a paclitaxel/steroid complex, oil for injection, water for injection, an emulsifier, an assistant emulsifier and an isotonic agent, wherein in said paclitaxel/steroid complex, the molar ratio between paclitaxel and steroid is 1:0.2˜4; preferably 1:0.2˜2; best 1:0.33˜1.

2. The paclitaxel submicron emulsion according to, claim 1 characterized in that, the said steroid in the paclitaxel/steroid complex is at least one of the natural steroids or their derivatives.

3. The paclitaxel submicron emulsion according to claim 1, wherein said natural steroid is selected from the group consisting of cholesterol, 7-hydrocholesterol, lanosterol, sitosterol, brassicasterol, mycosterol, ostreasterol, stigmasterol, sitosterolum and ergosterol; said natural steroid derivative is selected from the group consisting of cholic acid, deoxycholic acid and anthropodesoxycholic acid.

4. The submicron emulsion according to claim 1, wherein the average droplet diameter of said submicron emulsion is under 400 nm, the ratio of said oil phase is 5%˜35% (ml/ml), the drug load is 0.25 mg/ml˜5 mg/ml, preferably 0.5 mg/ml˜5 mg/ml if measured by paclitaxel; preferably, the average particle diameter of submicron emulsion droplet is under 300 nm, the ratio of said oil phase is 10%˜30% (ml/ml), the drug load is 0.5 mg/ml˜2 mg/ml, preferably 0.5 mg/ml˜5 mg/ml if measured by paclitaxel.

5. The submicron emulsion according to claim 1, wherein said oil for injection is one or mixture from long chain or medium chain oil; said long chain oil is selected from the group consisting of long chain fatty acid, long chain fatty ester or long chain fatty alcohol; the medium chain oil is selected from the group consisting of medium chain fatty acid, medium chain fatty ester and medium chain fatty alcohol.

6. The submicron emulsion according to claim 5, wherein the oil for injection is long chain fatty ester, said medium chain oil is medium chain fatty acid glyceride.

7. The submicron emulsion according to claim 1, wherein said emulsifier is a nonionic surfactant or natural surfactant; wherein said nonionic surfactant is selected from the group consisting of fatty acid glyceride, polyoxyethylene fatty alcohol ethers, polyoxyethylene sorbitan monoacid esters, sorbitol and sorbitan fatty acid ester, polyoxyethylene fatty acid ethers, vitamin E derivatives and polyoxyolefin copolymer; and wherein said natural surfactant is selected from the group consisting of egg yolk lecithin, fabaceous lecithin, ornitrol and cholic acids, sodium alginate and chitosan, preferably egg yolk lecithin and soybean lecithin.

8. The submicron emulsion according to claim 7, wherein the content of said emulsifier is 0%-5% (g/ml) of the total amount of the said submicron emulsion, preferably 1.0%-4.0% (g/ml), more preferably 1.0%-2.0% (g/ml).

9. The submicron emulsion according to claim 1, wherein said assistant emulsifier is selected from the group consisting of polyethyleneglycol (PEG) and poloxamer 188, preferably poloxamer 188.

10. The submicron emulsion according to claim 9, wherein the content of said assistant emulsifier is 0%-5% (g/ml) of the total amount of said submicron emulsion, preferably 0.5%-3% (g/ml), best 1.0%-2.0% (g/ml).

11. The submicron emulsion according to claim 1, wherein said isotonic agent is selected from the group consisting of glycerin, xylitol, sorbierite and mannitol, preferably glycerin.

12. The submicron emulsion of claim 1, wherein a stabilizer could also be included and said stabilizer is selected from the group consisting ofoleic acid, eunatrol and PEGs, preferably oleic acid.

13. The submicron emulsion of claim 1, wherein an antioxidant could also be included and said antioxidant is selected from the group consisting of vitamin E or vitamin E ester derivatives, preferably vitamin E.

14. A method for preparing a submicron emulsion of claim 1, which comprises the following steps:
preparing water for injection, adding an emulsifier, an assistant emulsifier and an isotonic agent, obtaining an even water phase by dispersing in a tissue disintegrator or shear, heating up to 40-80° C., and insulating the heated preparation;
mixing paclitaxel and steroid at a ratio as described, adding an appropriate volume of an organic solvent, agitating under a proper temperature, removing the organic solvent, obtaining the complex after vacuum drying; dissolving the obtained paclitaxel cholesterol complex and a stabilizer in the oil for injection preheated up to 40-80° C., and obtaining an even oil phase by dispersing in a tissue disintegrator or shear; and
under agitating, adding the water phase into the oil phase slowly, disintegrating for 5-10 min at 10000-20000 r/min to obtain a preliminary emulsion, and transferring the preliminary emulsion into a high pressure homogenizer quickly, obtaining particles with diameter under 400 nm through homogeneous emulsification, preferably under 300 nm, collecting all the emulsion, adjusting the pH to 3.5-6.0 with hydrochloric acid, preferably 4.0-5.0, and adding an appropriate amount of water, thereby obtaining the submicron emulsion product.

15. A method for preparing a submicron emulsion of claim 1, which comprises the following steps:
preparing water for injection, adding an assistant emulsifier and an isotonic agent, obtaining a water phase by agitating, heating up to 40-80° C., and insulating the heated preparation;
mixing paclitaxel and steroid at a ratio as described, adding an appropriate volume of an organic solvent, agitating under a proper temperature, removing the organic solvent, and obtaining a paclitaxel/steroid complex after vacuum drying; dissolving both the obtained paclitaxel/steroid complex and an emulsifier into the oil for injection preheated up to 40-80° C., obtaining an even oil phase by dispersing in a tissue disintegrator or shear;
under agitation, adding the water phase into the oil phase slowly, agitating and disintegrating at high speed to obtain a preliminary emulsion, and transfer the preliminary emulsion into a high pressure homogenizer quickly, obtaining particles with diameter under 400 nm through homogeneous emulsification, preferably 100-300 nm, collecting all the emulsion, adjusting the pH to 3.5-6.0 with hydrochloric acid, preferably 4.0-5.0, and adding an appropriate amount of water, thereby obtaining the submicron emulsion product.

16. A formulation, which comprises a paclitaxel submicron emulsion of claim 1 and is an infusion solution or dry emulsion.

17. A method for preparing the formulation according to claim 16, wherein the infusion solution is prepared by a procedure comprising: subjecting the paclitaxel submicron emulsion of claim 1 to an aseptic process performed through circulating steam sterilization or steam sterilization; wherein the dry emulsion is prepared by a process comprising: adding an appropriate amount of a support agent into the paclitaxel submicron emulsion of any one of claims 1-13, and subjecting the resulting preparation to an aseptic filtering, and obtaining the dry emulsion through freeze-drying.

18. Use of the paclitaxel submicron emulsion according to claim 1 or the formulation according to claim 16 in preparing drugs for treating oophoroma, breast cancer, cervical carcinoma, non-small cell lung cancer, head or neck cancer, esophagus cancer, renal carcinoma, liver cancer and gastric cancer.

19. Use of the formulation according to claim 16 in preparing drugs for treating oophoroma, breast cancer, cervical carcinoma, non-small cell lung cancer, head or neck cancer, esophagus cancer, renal carcinoma, liver cancer and gastric cancer.
